(19) United States
(12) Patent Application Publication
(10) Pub. No.: US 2005/0043280 A1
(43) Pub. Date: Feb. 24, 2005

(54) Use of active ingredients having a mu-opioid receptor agonist action and an opiod receptor antognist action, as combination drugs for the treatment of cancer

(76) Inventors: Gerd Geisslinger, Bad Soden (DE); Irmgard Tegeder, Frankfurt (DE)

Correspondence Address: FOLEY AND LARDNER;SUITE 500, 3000 K STREET NW, WASHINGTON, DC, 20007, US

(21) Appl. No.: 10/488,081

(22) PCT Filed: Jul. 23, 2002
(86) PCT No.: PCT/EP02/08181

(30) Foreign Application Priority Data
Sep. 1, 2001 (DE) 101429967

(51) Int. Cl. A61K 31/60; A61K 31/485; A61K 31/445

(52) U.S. Cl. 514/165; 514/282; 514/317

(57) ABSTRACT

The invention relates to the use of active ingredients having a μ-opioid receptor agonist action and an opioid receptor antagonist action, as combination drugs for the treatment of cancer.

Drugs for the treatment of cancerous diseases in humans and animals, said drugs containing opioid receptor-agonistic active ingredients and opioid receptor-antagonistic active ingredients either in a single or separate forms of administration and being applicable simultaneously or in a time-shifted fashion at adequate dosage for patient-optimized therapy.

In the industrialized countries, cancerous diseases are amongst the most common causes of death. The incidence, i.e. the frequency at which the disease occurs, depends on disposition (inclination to be affected), exposure (exposure to cancer-inducing influences such as chemicals, radiation, viruses, stress, etc.), and age. The increase in the incidence of cancerous diseases in the past century has been ascribed mainly to the increase of life expectancy. Due to the frequency and severity of this disease intensive research has been and continues to be devoted to the prevention, diagnosis, and therapy of cancer. Despite these efforts and the measures of early detection that have been undertaken, the probability of healing of the most common forms of cancer has improved only slightly, with some notable exceptions. All therapeutic measures are based on the removal or inhibition of growth of the degenerated cancer cells. Clinical options of reverting the development of a tumor cell to a normal tissue cell are not yet available. Depending on the specific type of the cancerous disease, the stage of disease, and the status of the patient, the available medical treatment procedures include surgery, irradiation, and the administration of medications. Drug therapy involves the use of inhibitors of mitosis, alkylating cytostatics, antibiotics with cytostatic effects, antimetabolites, hormones and hormone antagonists, immunomodulators, enzymes or radioisotopes to destroy or inhibit the growth of cancer cells. Methods for tissue-specific cancer therapy on the basis of drugs are still in the developmental phase as are measures involving gene therapy. Means of selective intervention in the signal transduction of the cell cycle of the cancer cell or angiogenesis (new formation of vessels) within the tumor are still being researched.

All medication-based cancer therapies known to date are based on a relatively unspecific intervention in the cell cycle of the cancer cell. Invariably, the drug attack also affects healthy cells such that, depending on the mechanism of action of the substance, the treatment is associated with various, usually substantial, adverse drug effects.

It was therefore the task of the present invention to provide active ingredients for cancer therapy, which active ingredients are known as drugs from other fields of application. These active ingredients should have similar desired effects on the tumor cell as the cancer therapeutics used until now, albeit at a more favorable side effect profile, if possible.

Due to the intensive international research efforts, in particular at the NCI (US National Cancer Institute), directed at identifying potential anti-cancer agents from the wealth of? available chemical and natural substances, the probability of identifying anticancer agents amongst the known drugs seemed very low. However, surprisingly this was achieved with the simultaneous application of the known pain-relieving agent (opioid), morphine, and the opioid antagonist, naloxone. Subsequent systematic investigations allowed the extension of this therapeutic concept to other active ingredients with similar mechanisms of action, i.e. opioids and opioid antagonists.

Receptor binding studies, cell culture experiments or animal experiments in the scientific literature provide some evidence to indicate that morphine and similar substances can intervene in the regulation of cell survival of non-tumor cells as well as in the biology of tumor cells and that opiate antagonists, such as naloxone or naltrexone, can suppress this effect [Polakiewicz, R. D., Schieferl, S. M., Gingras, A. C., Sonenberg, N. & Comb, M. J. “mu-opioid receptor activates signaling pathways implicated in cell survival and translational control” J Biol Chem 273, 23534-41 (1998); Maneckjee, R. & Minna, J. D. “Opioids induce, while nicotine suppresses, apoptosis in human lung cancer cells” Cell Growth Differ 5, 1033-40 (1994); Maneckjee, R. & Minna, J. D. “Non-conventional opioid binding sites mediate growth inhibitory effects of methadone on human lung cancer cells” Proc Natl Acad Sci USA 89, 1169-73 (1992); Zagon, I. S. & McLaughlin, P. J. “Heroin prolongs survival time and retards tumor growth in mice with neuroblastoma” Brain Res Bull 7, 25-32 (1981); Sueoka, N., Sueoka, E., Okabe, S. & Fujiki, H. “Anti-cancer effects of morphine through inhibition of tumor necrosis factor-alpha release and mRNA expression” Carcinogenesis 17, 2337-41 (1996)]. Since morphine or its related substances continue to be used very restrictively in the pain treatment of cancer patients, it appeared unjustified to use these substances for cancer therapy unless the patients also required this as co-therapy for pain. Especially its addictive potential and the depressive effect on the respiratory center were reasons not to use morphine or similar substances in patients who are free of pain or can be treated sufficiently with other painkillers, especially if tumor treatment necessitated elevated and thus more toxic dosages. The same literature references evidence that naloxone or other opioid antagonists, which reduce the toxicity of opioids, also suppress the tumor growth-inhibiting effect of the opioids in the lung tumor cell cultures investigated therein. Therefore, this pathway appeared unfeasible to increase the quantity of opioid. Surprisingly, though, whole animals were found to respond differently from what was suspected from the cell culture experiments.

The present invention is based on the following results.

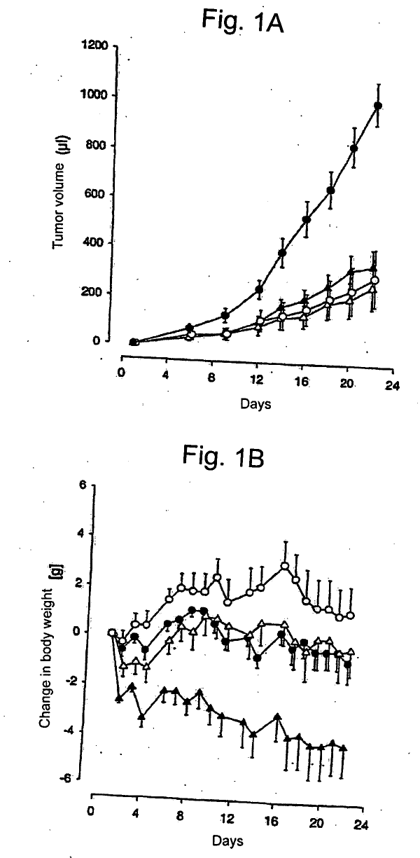

In an established pharmacological animal model, naked mice (NMRI-nu/nu; Harlan, Borchen) received a subcutaneous injection containing a cell suspension (5×10⁶ cells in 300 μl per mouse) of human breast carcinoma cells (MCF-7 cell line). The animals of the control group were treated for a period of 3 weeks with physiological saline and the animals of the three treatment groups for the same period of time with morphine, naloxone or a combination of morphine and naloxone (FIG. 1 with detailed description of the experimental design). The dose increase each week was intended to compensate for the known development of morphine tolerance. The naloxone dose was 1/10 of the morphine dose, since this dosage ratio is known to completely antagonize the effects of morphine. Compared to the control group, morphine treatment reduced tumor growth within 3 weeks to approx. one third, which was statistically highly significant (FIG. 1A). Surprisingly, the combination of morphine and the opioid antagonist, naloxone, showed at least the same, if not a superior, effect. Because of the complete antagonism of the typical pharmacological effects of morphine by naloxone at these dosages, these results can be interpreted to show that mechanisms of action other than the ones responsible for pain relieve make an essential contribution to the antitumor effect of morphine. FIG. 1B shows one known adverse effect, i.e. weight loss in the mice treated with morphine. In contrast, the body weight was not affected in the control group and in the group receiving morphine+naloxone treatment. The body weight of the animals in the naloxone group increased slightly.

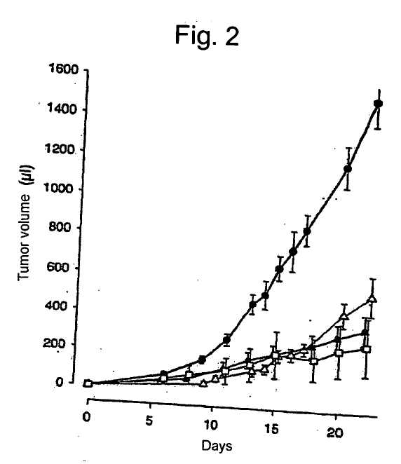

In another animal experiment with a similar design (FIG. 2) aiming to verify the surprising results, morphine, morphine-6-glucuronide (a morphine metabolite), and morphine+naltrexone (an opioid antagonist) in combination were compared to a control. Morphine was thus confirmed to have a good antitumor effect and morphine in combination with the opioid antagonist, naltrexone, was confirmed to possess a similar, if not superior, effect. Morphine-6-glucuronide proved to be similarly, but somewhat less effective, than morphine. The mechanism of the thus discovered advantageous effects was then investigated systematically in order to further elucidate the therapeutic relevance of these findings as well as their applicability to similar active ingredients.

The model substances used in the further investigations included DAMGO {(D-Ala2, N-Methyl-Phe4, Gly-ol5)enkephalin}, naloxone, and the combination of these two active ingredients. These substance are in common use in pharmacological research in order to elucidate the general principles of action of opioids. The term, opioids, includes all substances whose effect is mediated by binding to the opioid receptors in the body. This includes the large number of opium alkaloids from the seed capsules of Papaver somniferum, of which morphine is the most relevant for medicine. Other active substances can be prepared from the opium alkaloids by partially synthetic conversion. In the body, an inherent pain-inhibiting system produces opioid peptides, e.g. endorphins, enkephalins, and dynorphins. And lastly, there also are fully synthetic active ingredients whose pharmacological effect is mediated by binding to the opioid receptors.

Substances binding to opioid receptors may possess agonist (or agonistic=stimulating), antagonist (or antagonistic=inhibiting) or mixed agonist-antagonist effects. Because of these differences and due to the fact that the opioid receptors consist of multiple subtypes (μ, κ, and δ receptors) that are involved in different physiological functions, it is evident that substances binding to the opioid receptors may differ both in the quality and the intensity of their effects. With regard to their essential effects, though, all agonists and all antagonists show similar behavior. The model substance, DAMGO, that was selected for the further studies is a selective μ-agonist that is widely established in pharmacological research and is used to investigate the most significant pain-inhibiting effect of the opioids with regard to their clinical effects and for targeted study of the adverse effects of these substances. Experience tells that findings made with DAMGO can be extended to other opioids with a predominant μ-agonist effect. Naloxone is the opioid receptor antagonist that is most commonly used in pharmacological research and clinical applications. The substances acts antagonistic on all known opioid receptor subtypes.

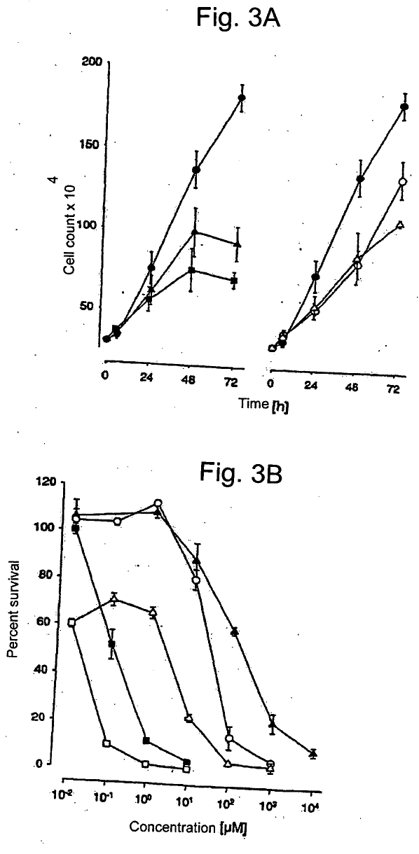

A cell proliferation test with human MCF-7 breast cancer cells was used to measure the effects of DAMGO, naloxone, and the mixture of these two active ingredients (FIG. 3A).

Compared to the untreated control, DAMGO reduced the cell division cycle substantially in a concentration-dependent fashion. In the DAMGO-treated cultures, the maximal number of cells was attained after 48 hours. Between 48 and 72 hours, a fraction of the cells on DAMGO treatment died (FIG. 3A, left side). Interestingly, treatments involving naloxone and the mixture of naloxone and DAMGO also showed a growth-inhibitory effect (FIG. 3A, right side). Consequently, the growth-inhibiting effect of DAMGO was not antagonized by naloxone.

The same cell line was used to determine the cytotoxic effects of these active ingredients by determining the rate of survival of the cells (FIG. 3B). DAMGO and, interestingly, naloxone again showed a concentration-dependent positive effect on the survival rate. When increasing concentrations of naloxone were added to 100 μmol DAMGO, the survival curve was clearly shifted towards the left. This shows not only that the mixture of the two active ingredients is substantially more effective than either of the substances alone, but also that there is no mutual antagonism between DAMGO and naloxone. TNFα was used as a positive control. The addition of DAMGO further enhanced the effect of TNFα to a substantial degree.

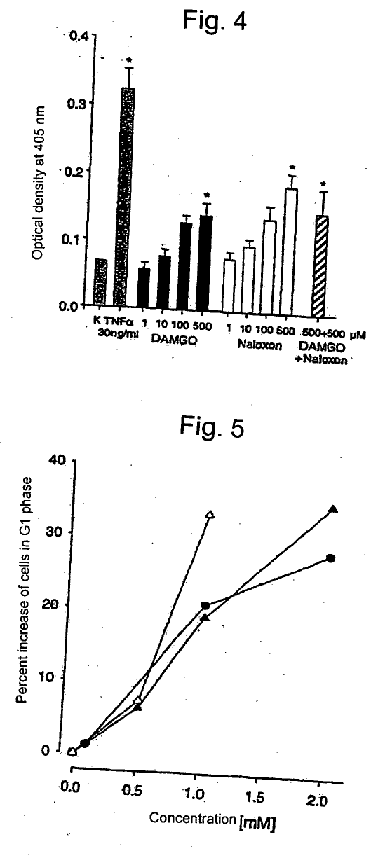

In order to characterize apoptosis (cell death) in more detail, a commercial apoptosis assay kit that is in common use in science was used to measure the rate of DNA cleavage (FIG. 4). TNFα is known to induce apoptosis in MCF-7 cells and was therefore used as a positive control. Compared to the untreated control, 500 μmol DAMGO, 500 μmol naloxone, and 500 μmol DAMGO+naloxone caused the rate of DNA cleavage to increase significantly.

Due to it being a peptide, the selective μ-agonist, DAMGO, is currently not in clinical use. In order to ensure that the results obtained with this substance are also applicable to the opioid agonist, morphine, which is most commonly used opioid in clinical applications showing a relatively selective μ-agonist effect, but also some weak binding to δ and κ opioid receptors, the cell cycle of MCF-7 cells was analyzed after treatment with morphine, naloxone or morphine+naloxone (FIG. 5). The measurement focused on the fraction of cells in G1 phase which is the phase before S-phase (DNA synthesis phase) in the interphase of the cell cycle. The concentration-dependent increase in the number of cells in G1 phase shows that morphine inhibits the progression of the cell cycle from G1 to S phase. Surprisingly, naloxone was observed to block the cell cycle in a similar fashion. The combination of the two active ingredients was more effective than either active ingredient alone, which is indicative of the existence of an additive effect in the combination.

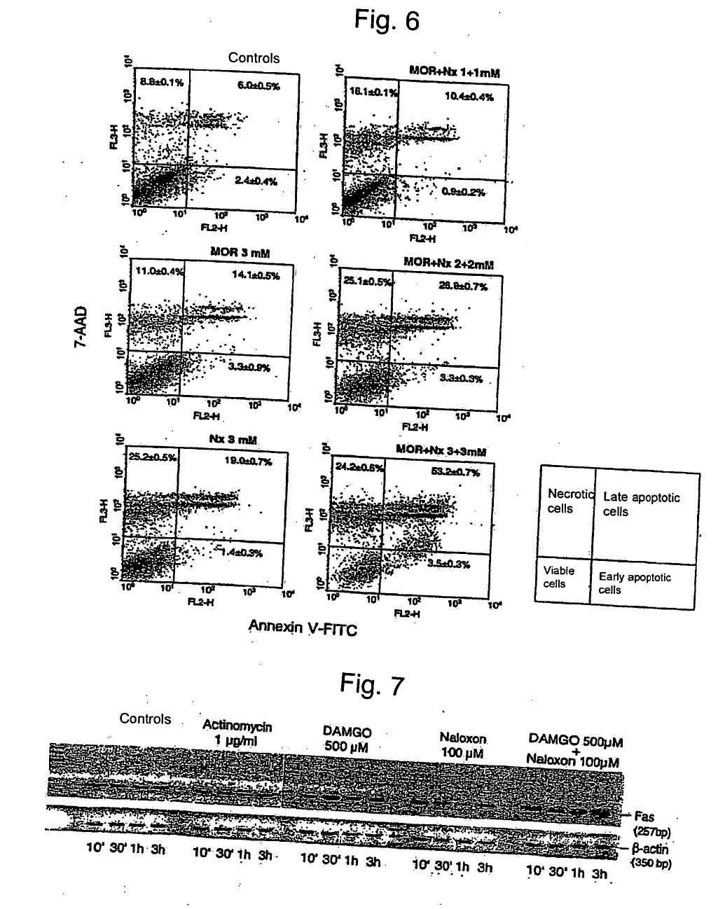

The effects of morphine, naloxone or the mixture of the two active ingredients on apoptosis was further investigated by analyzing, under the influence of these substances, the binding of annexin V to phosphatidylserine of the plasma membrane and of 7-amino-actinomycin D (7-AAD) to DNA by means of flow cytometry (FIG. 6). At concentrations of 3 mmol, both morphine and naloxone caused a clear increase in the number of cells in late apoptosis. This effect was less pronounced at 2 mmol and no longer detectable at 1 mmol (not shown). As before, the mixture of active ingredients was more effective than either active ingredient alone. Mutual antagonism between morphine and naloxone with regard to this effect did not occur.

From the investigations presented above as well as further studies according to the present invention, it can be concluded that active ingredients from the group of μ opioid receptor agonists, in particular the clinically relevant morphine, induce a blockage of the cell cycle and increase in the rate of cell death in the model experiment on human MCF-7 breast cancer cells. Similarly, tumor growth in naked mice is inhibited in the established pharmacological model with this cell line. The morphine concentrations in the plasma of these animals (not shown) corresponded to the plasma levels of patients on chronic oral administration of morphine for pain therapy. No substantial mutual antagonism with regard to the effects investigated was observed. The combination of agonist and antagonist was at least as effective as, or more effective than, either individual substance alone. Overall, the substances act through a partially μ-receptor-mediated mechanism including a phosphorylation and stabilization of p53 and up-regulation of p53-dependent proteins, including p21, Bax, and the “death receptor”, CD95/Fas (FIG. 7)(shown only in part). The absence of a mutual antagonism with regard to an essential fraction of the effects shown evidences that some of the effects are mediated by mechanisms that are independent of the known μ-receptor effects.

The investigations conducted according to the present invention show that μ opioid receptor agonists are suitable for use as active ingredients for cancer therapy. This holds true for both selective and partially selective μ agonists. By combining these active ingredients with opioid receptor antagonists it is possible to enhance the effects detected for the agonist or at least fully maintain these effects while reducing the typical side effects of opioid receptor agonists.

The application of combinations of μ opioid receptor agonists and opioid receptor antagonists allows the dosage ratios to be adjusted to the individual therapeutic needs. Patients experiencing no pain are treated with a combination of the agonist at an effective anticancer dose, whereby the fraction of the antagonist is selected such that the typical opioid effects are completely abolished. This allows the substantial side effects of this class of substances to be largely prevented. Therefore, due to the reduced or completely eliminated addictive potential, opioid receptor agonists, in particular the clinically relevant morphine, can be used in clinical applications without being limited by the otherwise common restrictions. Depending on the severity of the pain, the relative fraction of opioid receptor agonist can be increased such that, aside from a sufficient dose for cancer therapy, a sufficient pain-relieving effect is obtained by a relative dose excess of the agonist. The application of the combination allows the dose of the agonist to be selected higher if required by the clinical status, since the complete or partial abolishment of the μ receptor effect by the antagonist minimizes the typical side effects. Since the efficacy of cancer treatment is basically not subject to any mutual antagonism of the substances present in the combination, the agonist dose can be selected to be higher than in the corresponding monotherapy such that a maximal desired effect with clearly reduced side effects is obtained.

In cancer patients experiencing pain, the mixture of opioid receptor agonists and antagonists can be supplemented with other pain-relieving substances, such as paracetamol, acetylsalicylic acid, non-steroidal anti-inflammatory drugs, selective COX-2 inhibitors, etc. In many cases, these substances show an additive pain-relieving effect as compared to the application of opioid receptor agonists alone because their mechanism of action is different. If the pain-relieving effect is brought about by these additional components of the combination, the fraction of the antagonist in the drug can be increased relative to the fraction of agonist such that an optimal anti-cancer effect is achieved at minimal side effects and good relieve of pain.

The simplest option for the simultaneous application of l opioid receptor agonists and opioid receptor antagonists, possibly in combination with other pain-relieving medications, is to prepare the substances in the form of a single form of administration. By varying the dosage ratios of agonist and antagonist, finished pharmaceutical forms of administration to suit the individual therapeutic needs mentioned above can be prepared. Agonist, antagonist, and the additional pain-relieving agent, if any, can also be provided in the form of separate forms of administration. In the individual case, this would make the treatment even more optimized with regard to the needs of the individual patient, since both the dosage and the dosage interval can be selected individually.

The drugs can be administered according to any of the types of applications that are established in medicine. This includes for instances forms which are administered into the tumor by intravenous, intraarterial (e.g. into the artery supplying the tumor), intramuscular, subcutaneous, intraperitoneal, oral, buccal, rectal, topical, transdermal, epidural, intrathecal or local application. The possible preparations include e.g. solutions, suspensions, emulsions, tablets, capsules, potable preparations, suppositories, semi-solid preparations, transdermal therapeutic systems, and formulations for inhalation. These preparations are manufactured through the use of excipients that are in common use in pharmaceutical technology. The available extensive expertise on the biopharmaceutical and pharmacokinetic properties as well as on the common medical application of these well-known individual substances can be applied in order to optimize both the preparation and the application of these drugs.

The following figures provide a more detailed illustration of the present invention. It is shown in:

FIG. 1A MCF-7 tumor growth in NMRI nu/nu naked mice (Harlan, Borchen) after treatment with morphine (solid triangles; n=15), naloxone (open circles; n=8), morphine+naloxone (open triangles; n=11) or vehicle (solid circles; n=13). A total of 5×10⁶ cells were injected subcutaneously per mouse. The morphine and naloxone doses were 10, 20, and 30 mg/kg daily in week 1, 2, and 3. The active ingredient combination contained daily morphine doses of 10, 20, and 30 mg/kg and naloxone doses of 1, 2, and 3 mg/kg. The animals were treated with 10 mg/kg β-estradiol valerate (i.m.) one day before and 7 and 14 days after the injection of tumor cells. This substance promotes tumor growth and is thus in standard use in this animal model. Tumor volume=(long diameter×short diameter²)/2.

FIG. 1B Development of the body weight during treatment. Treatment as in 1A.

FIG. 2 MCF-7 tumor growth in NMRI nu/nu naked mice after treatment with vehicle (control; n=15), morphine (n=10), morphine-6-glucuronide (n=5) or morphine+naltrexone (n=5). A total of 4×10⁶ MCF-7 cells were injected subcutaneously on day 0. The following daily doses were administered: morphine: 10, 15 or 20 mg/kg in week 1, 2 and 3, respectively; morphine-6-glucuronide: 10 mg/kg; morphine+naltrexone: 10+10, 15+15 or 20+20 mg/kg in week 1, 2 or 3, respectively.

FIG. 3A Proliferation of MCF-7 cells in vitro after treatment with 100 μmol DAMGO (solid triangles), 500 μmol DAMGO (solid squares)(left), 100 μmol naloxone (open circles) or the mixture containing 500 μmol DAMGO+100 μmol naloxone (open triangles)(right) versus untreated control cells (solid circles). Means±SEM from 4-6 repeat experiments.

FIG. 3B Survival rate of MCF-7 cells after treatment with DAMGO (solid triangles), naloxone (open circles), 100 μmol DAMGO+naloxone (open triangles), TNFα (solid squares), and 100 μmol DAMGO+TNFα (open squares). The survival rate was standardized to the number of untreated control cells representing 100%. Means±SEM from 4 repeat experiments.

FIG. 4 DNA cleavage rate after incubation of MCF-7 cells for 24 hours with the active ingredients at the dosages indicated on the abscissa. Means±SEM from 6 independent experiments. DNA cleavage was measured by assaying the mono- and oligonucleosomes with an apoptosis detection kit containing antihistones and anti-DNA antibodies.

FIG. 5 Analysis of the cell cycle by FACS analysis of MCF-7 cells 24 hours after treatment with morphine (solid triangles) or naloxone (solid circles) at concentrations of 0.5; 1.0; and 2.0 mmol and after treatment with morphine+naloxone (open triangles) at concentrations of 0.5 mmol morphine+0.1 mmol naloxone and 1 mmol morphine+1 mmol naloxone. Means from 3 independent experiments.

FIG. 6 Flow cytometry analysis of annexin V and 7-AAD binding to determine the apoptosis rate 24 hours after treatment with morphine (NOR), naloxone (Nx) or the combination of morphine and naloxone at the indicated concentrations.

FIG. 7 Expression of CD95/Fas mRNA in MCF-7 cells after treatment with actinomycin D (positive control), DAMGO, naloxone or DAMGO+naloxone at the concentrations and for the times indicated. The Fas-mRNA content was assayed by rt-PCR using 1 μg total RNA. Representative results from 3 independent experiments.

1. Use of active ingredients having a μ-opioid receptor agonist action and opioid receptor antagonist action, as combination drugs for the treatment of cancer.

2. Use of active ingredients having a μ-opioid receptor agonist action and opioid receptor antagonist action, as combination drugs for the treatment of breast cancer.

3. Use according to claim 1, characterized in that the μ-opioid receptor agonists and opioid receptor antagonists are combined into a single form of administration or prepared individually and supplied for therapy in a combination package or prepared individually and supplied for therapy in separate drug packages.

4. Use according to claim 1, characterized in that the ratio of the μ-opioid receptor agonists and opioid receptor antagonists is adjusted such that the typical pain-related effects and side effects of the opioid receptor agonist are completely antagonized by the opioid receptor antagonist.

5. Use according to claim 1, characterized in that the ratio of the μ-opioid receptor agonists and opioid receptor antagonists is adjusted such that the fraction of the μ-opioid receptor agonist is larger than the fraction of the opioid receptor antagonist.

6. Use according to claim 1, characterized in that the μ-opioid receptor agonists and opioid receptor antagonists are supplemented with other pain-relieving or anti-inflammatory active ingredients, such as e.g. paracetamol, acetylsalicylic acid, non-steroidal anti-inflammatory drugs, selective COX-2 inhibitors, TNFα inhibitors, etc.

7. Use according to claim 1, characterized in that morphine, morphine-6-glucuronide, methadone, pethidine, fentanyl, tramadol, pentazocine, buprenorphine, tilidine, levomethadone, piritramide, levacetylmethadole, codeine, oxycodone, hydromorphone, dihydrocodeine, diamorphine, sufentanil, alfentanil, remifentanil, nalbuphine, butorphanol, the salts and derivatives of these substances, in particular the glucuronides, are used as the opioid receptor agonists.

8. Use according to claim 1, characterized in that naloxone or naltrexone are used as the opioid receptor antagonists.

9. Use according to claim 1, characterized in that the dosage of the opioid agonists exceeds their common dosage in the known clinical applications.

10. Use according to claim 1, characterized in that the active ingredients are used together with pharmaceutically and pharmacologically compatible excipients to manufacture finished pharmaceutical products, such as solutions for injection or infusion, suspensions, emulsions, tablets, capsules, potable preparations, chewable formulations, suppositories, semi-solid preparations, transdermal therapeutic formulations or formulations for inhalation.

11. Use according to claim 1, characterized in that the active ingredients are used together with pharmaceutically and pharmacologically compatible excipients to manufacture transdermal therapeutic systems with long-term continuous or intermittent release characteristics, in particular those containing the active ingredients, fentanyl and buprenorphine.

12. Use according to claim 1, characterized in that the drugs can be applied into the tumor by intravenous, intraarterial, intramuscular, subcutaneous, intraperitoneal, oral, buccal, nasal, rectal, topical, transdermal, epidural, intrathecal or local application.
